(19) United States
(12) Patent Application Publication
(10) Pub. No.: US 2023/0064362 A1
(43) Pub. Date: Mar. 2, 2023

(54) HIGH COHESIVE STRENGTH HARD COATINGS CONTAINING SOFT METAL

(71) Applicant: Oerlikon Surface Solutions AG, Pfäffikon, Pfäffikon (CH)

(72) Inventors: Martin DRABIK, Sargans (CH); Noora MANNINEN, Chur (CH); Canet ACIKGOZ DOROKIN, Zürich (CH)

(21) Appl. No.: 17/792,928

(22) PCT Filed: Jan. 12, 2021
(86) PCT No.: PCT/EP2021/000002

§ 371 Date: Jul. 14, 2022

Related U.S. Application Data
(60) Provisional application No. 62/961,291, filed on Jan. 15, 2020.

(51) Int. Cl. C23C 14/04; C23C 14/02; C23C 14/06; C23C 14/18

(52) U.S. Cl. CPC C23C 14/04; C23C 14/024; C23C 14/0641; C23C 14/18

(57) ABSTRACT

A method for introducing a soft metal into a hard coating during a physical vapor deposition process. The method including steps of providing a substrate; depositing a bonding layer on the substrate; and depositing the hard coating on the bonding layer using vapor deposition wherein the soft metal forms islands in the hard coating.

### FIELD OF THE INVENTION

The present invention relates to a hard coating, (e.g. metal nitrides, carbides, oxides or combination of those (e.g. metal carbonitrides) being the metal Ti, Zr, Ta, Al, Si, Cr, or a combination of those metals (e.g. TiN or TiZrN)), containing a soft metal (Ag, Au, Cu, Zn).

The present invention relates furthermore to a method for producing such soft metal containing hard coatings layers.

### BACKGROUND OF THE INVENTION

It is well known that soft metal (e.g. Ag, Au, Cu, Zn) containing hard coatings produced by physical vapor deposition (PVD) techniques can exhibit antibacterial activity; furthermore, the addition of soft metals to hard coatings can enhance the tribological properties due to the reduction of friction coefficient promoted by the presence of soft metals like Ag, Au, Cu and Zn. It is also known that incorporation of soft metals can increase the electrical conductivity of hard coatings, while keeping the wear and mechanical properties characteristic from hard coatings. Hard coatings containing Ag are the most well-known and widely described in literature.

It is also known to have an amount of Ag to be incorporated in the coating within a range of about 2 at. % to 15 at. %, as described by Colmenares Mora et al in U.S. Pat. No. 10,143,196B2, in order to obtain acceptable coating adhesion, coating hardness and coating roughness values. In addition the incorporation of Ag above 15 at. % results in a change in coating color, which in some applications (e.g. surgical instruments) is not a desired effect.

### SUMMARY OF THE INVENTION

An objective of the present invention is to provide a method involving PVD techniques for producing soft metal (Ag, Au, Cu, Zn) containing hard coatings with enhanced cohesive strength, which is of major importance for applications where high load-bearing capacity or protection against wear is required.

The present invention provides method for introducing a soft metal into a hard coating during a physical vapor deposition process. The method including steps of providing a substrate; depositing a bonding layer on the substrate; and depositing the hard coating on the bonding layer using vapor deposition wherein the soft metal forms islands in the hard coating.

In the foregoing method, the hard coating is TiN and the metal is Ag.

In the foregoing method, the coating comprises an AgTiN coating and a top layer comprising TiN.

In the foregoing method, the Ag is deposited in islands in the AgTiN coating.

### BRIEF DESCRIPTION OF THE DRAWINGS

Preferred embodiments are disclosed and described in detail herein with reference to the accompanying drawings which form a part hereof, and wherein:

FIG. 1 is a schematic representation of a coating according to the present invention;

FIG. 2A is a SEM cross-sectional micrograph of a layer like AgTiN (LL-AgTiN) in total cross-section;

FIG. 2B is a SEM cross-sectional micrograph of a layer like AgTiN (LL-AgTiN) providing a detail view of LL-AgTiN layer of FIG. 2a;

FIG. 3A is a STEM cross-sectional micrograph of a layer like AgTiN (LL-Ag—TiN) showing an overall view of LL-AgTiN and top TiN layer;

FIG. 3B is a STEM cross-sectional micrograph of a layer like AgTiN (LL-Ag—TiN) showing a detailed view of LL-AgTiN layer with identification of chemical compounds from EDS analysis;

FIG. 4A is a SEM cross-sectional micrograph of an island like AgTiN (IL-AgTiN) illustrated in total cross-section;

FIG. 4B is a SEM cross-sectional micrograph of an island like AgTiN (IL-AgTiN) illustrating a detail view of the IL-AgTiN layer of FIG. 4A;

FIG. 5A is a STEM cross-sectional micrograph of a layer like AgTiN (IL-Ag—TiN) showing an overall view of IL-AgTiN and top TiN layer;

FIG. 5B is a STEM cross-sectional micrograph of a layer like AgTiN (IL-Ag—TiN) showing a detailed view of IL-AgTiN layer with identification of chemical compounds from EDS analysis;

FIG. 6A is a Rockwell test (HRC Rockwell value) performed on an alloyed cold-work steel. 1.2842 on LL-AgTiN;

FIG. 6B is a Rockwell test (HRC Rockwell value) performed on an alloyed cold-work steel. 1.2842 on IL-AgTiN;

FIG. 7A is a light optical micrograph of a scratch test indents on layer-like AgTiN (LL-AgTiN) coating on alloyed cold-work steel 1.2842 illustrating a first spallation at 39 N;

FIG. 7B is a light optical micrograph of a scratch test indents on layer-like AgTiN (LL-AgTiN) coating on alloyed cold-work steel 1.2842 illustrating a scratch end at 60 N;

FIG. 8A is a light optical micrograph of a scratch test indents on layer-like AgTiN (IL-AgTiN) coating on alloyed cold-work steel 1.2842 illustrating a first spallation at 35 N;

FIG. 8B is a light optical micrograph of a scratch test indents on layer-like AgTiN (IL-AgTiN) coating on alloyed cold-work steel 1.2842 illustrating a scratch end at 60 N;

### DESCRIPTION OF PREFERRED EMBODIMENT

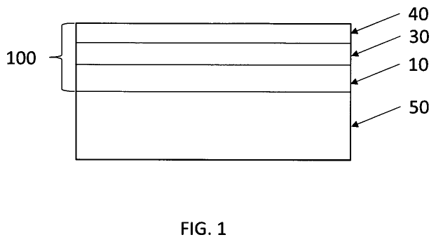

Referring now to the drawings, FIG. 1 shows a schematic representation of a coating according to the present invention wherein a coating 100 is applied to a substrate 50. It is contemplated that besides the amount of Ag incorporated in the coating 100, also the distribution of Ag within the coating 100 plays a major role in coatings properties.

The present invention, in general, provides a coating method for producing soft metal (Ag, Au, Cu, Zn) containing hard coatings with enhanced cohesive strength, which is obtained by tailoring the soft metal distribution within the hard coating.

In the embodiment described herein, the coating 100 is a AgTiN coating, comprising three major layers: a bonding layer 10 coated between the substrate 50 and an AgTiN coating 30, the AgTiN layer 30 and a top layer comprising TiN 40, as illustrated in FIG. 1.

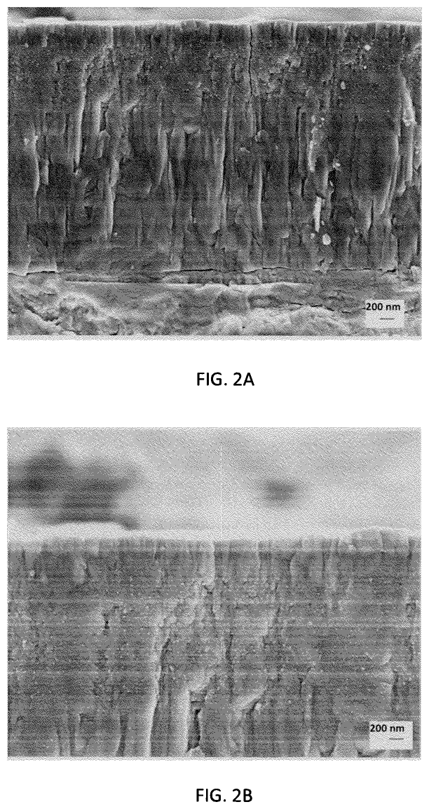

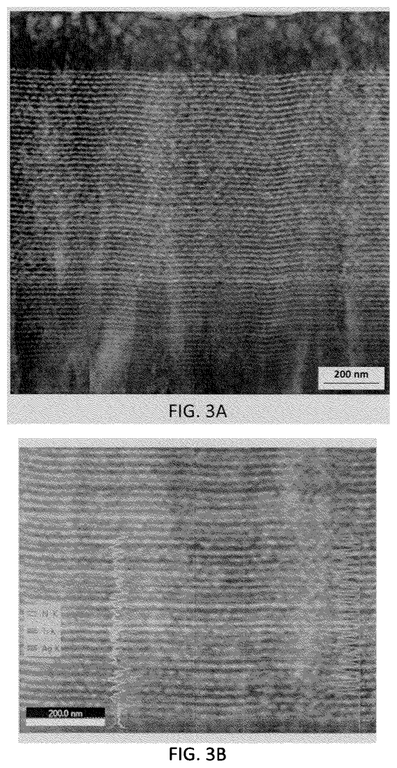

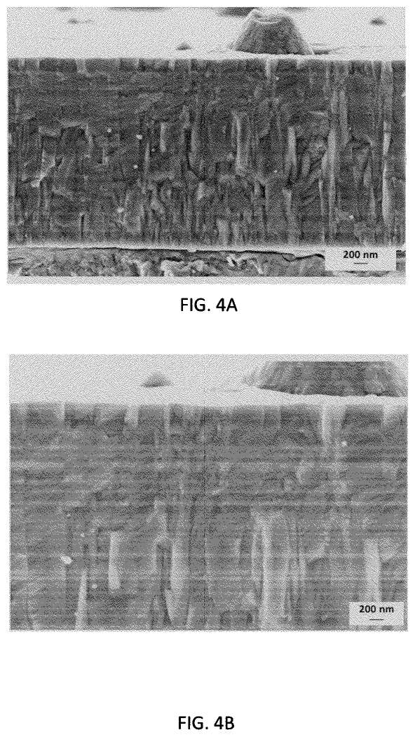

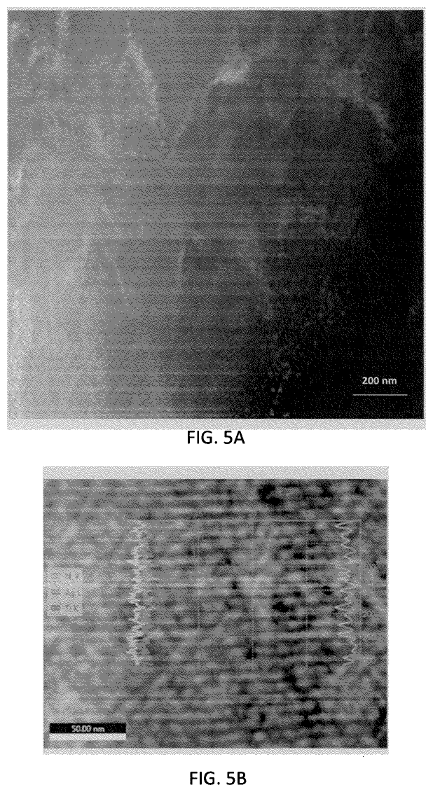

The growth of Ag structures within a hard coating can be tailored: (i) in order to allow formation of continuous Ag layers (see FIGS. 2A and 2B and FIGS. 3A and 3B) or (ii) in order to allow the formation of Ag islands (see FIGS. 4A and 4B and FIGS. 5A and 5B). The coating on FIGS. 2A and 2B and FIGS. 3A and 3B is termed as layer-like AgTiN (“LL-AgTiN”) due to the formation of continuous Ag layers alternating with continuous TiN layers. An estimated thickness of each individual Ag layer is on the order of 10 nm, as determined in scanning transmission electron microscopy/electron dispersive spectroscopy profiles (STEM/EDS) analyses. The presence of Ag layers can be already detected in scanning electron microscopy (SEM) cross-sectional analysis. In FIGS. 2A and 2B, the Ag is shown as the brighter layers/spots. The formation of Ag layers is more clearly observed in STEM analyses (FIGS. 3A and 3B), where Ag layers can be seen as brighter layers in relation to TiN, due to higher atomic mass of Ag. The presence of Ag layers can also be easily identified in FIG. 3B with support of EDS profiles.

The coating in FIGS. 4A and 4B and FIGS. 5A and 5B is termed as island like AgTiN (“IL-AgTiN”) due to the formation of Ag islands, instead of continuous Ag layers. Contrary to LL-AgTiN, no signs of Ag are visible in SEM analysis. The AgTiN layer shows a more compact structure and morphology due to the impact of Ag on TiN growth, however no Ag layers or large particles are visible in SEM analysis. The presence of Ag is detected only by STEM analysis in FIGS. 5A and 5B. In FIG. 5A the presence of Ag in layered structure is visible, however much less pronounced in comparison to LL-AgTiN shown in FIG. 3A. The Ag islands become more visible in FIG. 5B. For IL-AgTiN the Ag only becomes visible at very high magnifications, indicating that the Ag does not form continuous layers but islands. The main difference between the LL-AgTiN and IL-AgTiN is that the Ag inclusions form continuous layers in case of LL-AgTiN, while in IL-AgTiN the layer formation is interrupted in order to avoid the full coalescence of Ag and consequent formation of continuous Ag layers.

If, as described above the Ag layer formation is interrupted and islands are formed the main consequence is detected in coating cohesive strength, which is highly improved on island-like IL-AgTiN coating version as compared to layer-like AgTiN (LL-AgTiN).

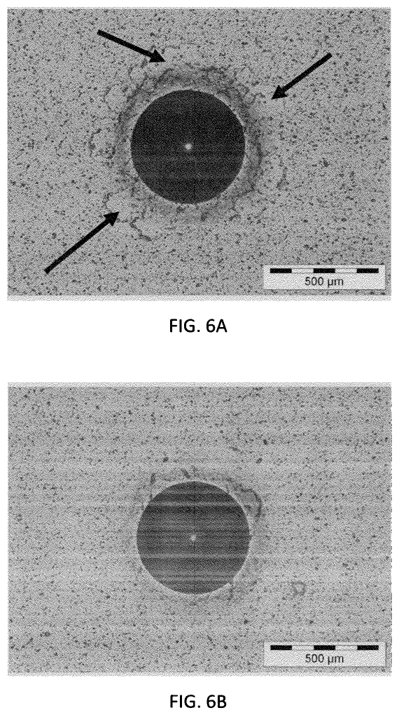

FIGS. 6A and 6B show light optical micrographs (LOM) of LL-AgTiN coating (FIG. 6A) and IL-AgTiN coating (FIG. 6B) after HRC Rockwell test, performed on alloyed cold-work steel 1.2842, following guidelines 3198 and 3824-4 of the VDI (Association of German Engineers).

The Rockwell indentation tests shown in FIGS. 6A and 6B show that no adhesive failure (detachment from substrate) is observed in any of the coatings. The coating with layer-like growth (LL-AgTiN) shown in FIG. 6A shows large areas with cohesive flaking in the edge area of the indentation, as indicated by the arrows. Conversely, no cohesive flaking is observed in island-like AgTiN (IL-AgTiN) coating shown in FIG. 6B.

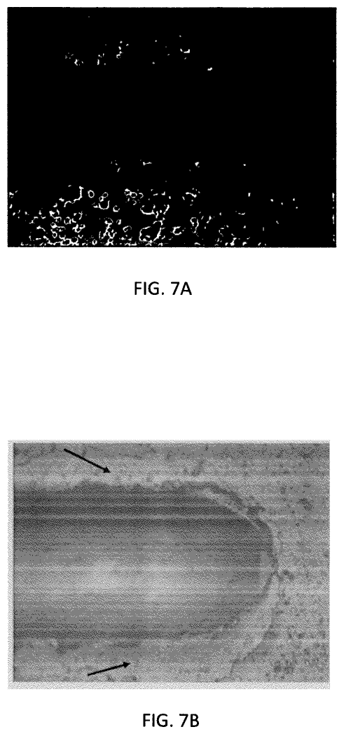

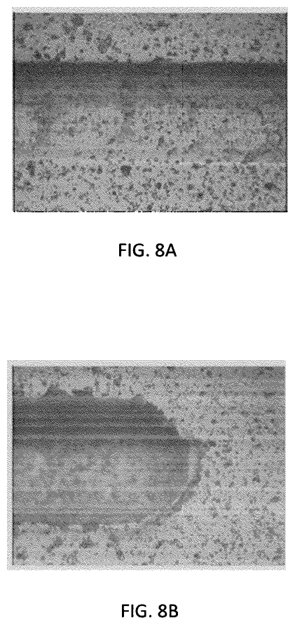

FIGS. 7A and 7B and FIGS. 8A and 8B show light optical micrographs (LOM) of LL-AgTiN coating (FIGS. 7A and 7B) and IL-AgTiN coating (FIGS. 8A and 8B) after scratch testing, performed on alloyed cold-work steel 1.2842, following guidelines ASTM C1624-05. Scratch testing was performed with an incremental load, starting at 5 N and ending at 60 N, applied at 100 N/min with a speed of 10 mm/min. A diamond indenter with radius of 200 μm was used. Six measurements were performed per coating in order to get statistical analysis.

Both coatings LL-AgTiN (FIGS. 7A and 7B) and IL-AgTiN (FIGS. 8A and 8B) show similar values of critical loads with Lc2 at 38.9±2.8 N for LL-AgTiN and 35.3±3.1 N for IL-AgTiN, being the Lc3>60N for both coatings. Despite no visible delamination of the coating from the substrate, LL-AgTiN shows high areas of cohesive flaking at the end of the scratch, which is not visible on IL-AgTiN, again indicating a higher cohesive strength on island-like AgTiN coating in comparison to layer-like AgTiN.

The deposition of hard coatings (e.g. TiN) containing soft metal (e.g. Ag) can result in different distribution of soft metal in hard coating. The distribution of soft metal within the hard coating plays a major role on coating cohesive strength. The growth of soft metal in island-like structure allow to improve the cohesive strength of the coating. Conversely the formation of continuous soft metal layers alternating with hard layers can result in poor cohesive strength, especially in cases where the soft metal does not form strong bonds with the hard coating.

A hard coating containing an island-like soft metal inclusions can be particularly suitable for applications where antibacterial activity (provided by the soft metal) and resistance to wear under high loads is required (e.g. articulating medical implants). The increase of electrical conductivity is also an advantage in some applications (e.g. sensors) where the wear and mechanical properties of hard coatings are required.

Illustrative embodiments have been described, hereinabove. It will be apparent to those skilled in the art that the above apparatuses and methods may incorporate changes and modifications without departing from the scope of this disclosure. The invention is therefore not limited to particular details of the disclosed embodiments, but rather encompasses the spirit and the scope thereof as embodied in the appended claims.

1. A method for introducing a soft metal into a hard coating during a physical vapor deposition process, said method comprising:
providing a substrate;
depositing a bonding layer on the substrate; and
depositing the hard coating on the bonding layer using vapor deposition wherein the soft metal forms islands in the hard coating.

2. The method of claim 1, wherein the hard coating is TiN and the soft metal is Ag.

3. The method of claim 2, wherein the hard coating comprises an AgTiN coating and a top layer comprising TiN.

4. The method of claim 3, wherein the Ag is deposited in islands in the AgTiN coating.
